(19) United States
(12) Patent Application Publication
(10) Pub. No.: US 2004/0034409 A1
(43) Pub. Date: Feb. 19, 2004

(54) Stent with polymeric coating

(75) Inventors: Bernd Heublein, Hannover (DE); Katrin Sternberg, Rostock (DE); Michael Tittelbach, Nuernberg (DE)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co.

(21) Appl. No.: 10/639,225

(22) Filed: Aug. 11, 2003

(30) Foreign Application Priority Data
Aug. 13, 2002 (DE) 102 37 572.0

(51) Int. Cl. A61F 2/06

(52) U.S. Cl. 623/1.46; 427/2.25

(57) ABSTRACT

The invention concerns an implantable stent (10) with an at least portion-wise polymeric coating (16). The coating material is admittedly intended to bond to known materials but by virtue of its properties it is intended to enjoy improved compatibility and reduce inflammatory and proliferative processes which can lead to restenosis. That is achieved in that the polymeric coating (16) in the implantable condition after production and sterilization contains poly-L-lactide of a mean molecular weight of more than 200 kDa.

The invention concerns an implantable stent with an at least portionwise polymeric coating and an associated process for the production of stents coated in that way.

### BACKGROUND OF THE ART

One of the most frequent causes of death in Western Europe and North America is coronary heart diseases. According to recent knowledge, in particular inflammatory processes are the driving force behind arteriosclerosis. The process is supposedly initiated by the increased deposit of low-density lipoproteins (LDL-particles) in the intima of the vessel wall. After penetrating into the intima the LDL-particles are chemically modified by oxidants. The modified LDL-particles in turn cause the endothelium cells which line the inner vessel walls to activate the immune system. As a consequence monocytes pass into the intima and mature to macrophages. In conjunction with the T-cells which also enter inflammation mediators such as immune messenger substances and proliferatively acting substances are liberated and the macrophages begin to receive the modified LDL-particles. The lipid lesions which are formed from T-cells and the macrophages which are filled with LDL-particles and which by virtue of their appearance are referred to as foam cells represent an early form of arteriosclerotic plaque. The inflammation reaction in the intima, by virtue of corresponding inflammation mediators, causes smooth muscle cells of the further outwardly disposed media of the vessel wall to migrate to under the endothelium cells. There they replicate and form a fibrous cover layer from the fiber protein collagen, which delimits the subjacent lipid core of foam cells from the blood stream. The deep-ranging structural changes which are then present in the vessel wall are referred to in summary as plaque.

Arteriosclerotic plaque initially expands relatively little in the direction of the blood stream as the latter can expand as a compensation effect. With time however there is a constriction in the blood channel (stenosis), the first symptoms of which occur in physical stress. The constricted artery can then no longer expand sufficiently in order better to supply blood to the tissue to be supplied therewith. If it is a cardiac artery that is affected, the patient frequently complains about a feeling of pressure and tightness behind the sternum (angina pectoris). When other arteries are involved, painful cramps are a frequently occurring sign of the stenosis.

The stenosis can ultimately result in complete closure of the blood stream (cardiac infarction, stroke). Recent investigations have shown however that this occurs only in about 15 percent of cases solely due to plaque formation. Rather, the progressive breakdown of the fibrous cover layer of collagen, which is caused by certain inflammation mediators from the foam cells, seems to be a crucial additional factor. If the fibrous cover layer tears open the lipid core can come directly into contact with the blood. As, as a consequence of the inflammation reaction, tissue factors (TF) are produced at the same time in the foam cells, and these are very potent triggers of the coagulation cascade, the blood clot which forms can block off the blood vessel.

Non-operative stenosis treatment methods were established more than twenty years ago, in which inter alia the blood vessel is expanded again by balloon dilation (PTCA—percutaneous transluminal coronary angioplasty). It will be noted however that expansion of the blood vessel gives rise predominantly to injuries, tears and disselections in the vessel wall, which admittedly heal without any problem but which in about a third of cases, due to triggered cell growth, result in growths (proliferation) which ultimately result in renewed vessel constriction (restenosis). The expansion effect also does not eliminate the physiological causes of the stenosis, that is to say the changes in the vessel wall. A further cause of restenosis is the elasticity of the expanded blood vessel. After the balloon is removed the blood vessel contracts excessively so that the vessel cross-section is reduced (obstruction, referred to as negative remodeling). The latter effect can only be avoided by the placement of a stent. The use of stents admittedly makes it possible to achieve an optimum vessel cross-section, but the use of stents also results in very minor damage which can induce proliferation and thus ultimately can trigger restenosis.

In the meantime extensive knowledge has been acquired in regard to the cell-biological mechanism and to the triggering factors of stenosis and restenosis. As already explained above restenosis occurs as a reaction on the part of vessel wall to local damage as a consequence of expansion of the arteriosclerotic plaque. By way of complex active mechanisms lumen-directed migration and proliferation of the smooth muscle cells of the media and the adventitia is induced (neointimal hyperplasy). Under the influence of various growth factors the smooth muscle cells produce a cover layer of matrix proteins (elastin, collagen, proteoglycans) whose uncontrolled growth can gradually result in constriction of the lumen. Systematically medicinal therapy involvements provide inter alia for the oral administration of calcium antagonists, ACE-inhibitors, anti-coagulants, anti-aggregants, fish oils, anti-proliferative substances, anti-inflammatory substances and serotonin-antagonists, but hitherto significant reductions in the restenosis rates have not been achieved in that way.

For some years, endeavors have been made to reduce the risk of restenosis in the implantation of stents by the application of special coatings. In part the coating systems themselves serve as a carrier matrix, in which one or more drugs are embedded (local drug delivery). In general the coating covers at least a surface, which is towards the vessel wall, of the endovascular implant.

The coatings almost inevitably consist of a biocompatible material which is either of natural origin or can be obtained synthetically. Particularly good compatibility and the possibility of influencing the elution characteristic of the embedded drug are afforded by biodegradable coating materials. Examples in regard to the use of biodegradable polymers are cellulose, collagen, albumin, casein, polysaccharides (PSAC), polylactide (PLA), poly-L-lactide (PLLA), polyglycol (PGA), poly-D,L-lactide-co-glycolide (PDLLA/PGA), polyhydroxybutyric acid (PHB), polyhydroxyvaleric acid (PHV), polyalkylcarbonate, polyothoester, polyethyleneterephthalate (PET), polymalic acid (PML), polyanhydrides, polyphosphazenes, polyamino acids and their copolymers as well as hyaluronic acid and derivatives thereof.

In the meantime numerous studies have demonstrated the positive effect of biocompatible coatings on the tendency to restenosis in the case of metallic stents. In spite of those successes there is still a not inconsiderable residual risk in terms of restenosis formation with the materials used hitherto. It is precisely the particularly inexpensive polylactides which are easy to process and which are particularly suitable as a polymer matrix for accommodating drugs that exhibit a detrimental inflammatory stimulus on the tissue environment, when using batches of conventional quality and molecular weight.

For most technical uses polylactides with molar masses in the range of between about 60 and 200 kDa are used (see for example H. Saechtling; Kunststoff Taschenbuch; (“Plastics Handbook); 28th edition; page 611). In a corresponding manner the polylactides used hitherto in the medical area of implantation technology have also been selected from that molar mass range.

To reduce the thrombogenic qualities of stents, a polylactide (PDLLA) coated stent with an embedded thrombin inhibitor has been proposed (Hermann R., Schmidmaier G., Märkl B., Resch A., Hahnel I., Stemberger A., Alt E.; Antithrombogenic Coating of Stents Using a Biodegradable Drug Delivery Technology; Thrombosis and haemostasis, 82 (1999) 51-57). The polymer matrix of PDLLA used was of a mean molecular weight of about 30 kDa. A coating of a thickness of about 10 μm of the same polymer material served in accordance with another study as a carrier for the active substances hirudin and iloprost (Alt E., Hähnel I. et al; Inhibition of Neointima Formation After Experimental Coronary Artery Stenting; Circulation, 101 (2000) 1453-1458).

In accordance with a further study, inter alia, PLLA of a molar mass of about 321 kDa was used for coating a coronary stent. In further investigations dexamethasone as an active substance was added to the polymer matrix. Sterilization was effected using the conventional ethylene oxide procedure. The coated stents were implanted in pigs and after 28 days histological analysis of neointimal hyperplasy was effected (Lincoff A. M., Furst J. G., Ellis S. G., Tuch R. J., Topol E. J.; Sustained Local Delivery of Dexamethasone by a Novel Intravascular Eluting Stent to Prevent Restenosis in the Porcine Coronary Injury Model; Journal of the American College of Cardiology, 29 (1997), 808-816).

German patent DE 198 43 254 describes the use of a blend of poly-L-lactide (batch L104 from Boehringer Ingelheim) and polycyanacrylic acid ester or polymethylene malic acid ester as a coating material for implants. According to the manufacturer's specifications the stated batch is of a mean molecular weight of about 2 kDa. U.S. Pat. No. 6,319,512 also discloses an implant for active substance delivery, the casing of which comprises a blend of poly-L-lactide of batch 104 and a copolymer of lactide and glycol.

Now and again the inflammatory action of poly-L-lactide is to be used to stimulate tissue re-formation. Thus United States published application No 2002/0040239 proposes, in the case of tissue injuries which heal poorly, introducing into the tissue small implants of inter alia poly-L-lactide. No details regarding the molar mass of the polymer are specified so that it is evidently assumed that a poly-L-lactide of the usual composition is sufficient to produce the effect.

The use of poly-L-lactides as a material for stents has also been described. Thus, in published European application 0 574 474, in amorphous/crystalline polymer mixtures with a plasticiser, in U.S. Pat. No. 6,368,346 as a constituent of a blend and in clinical studies on a human being (Tsuji T., Tamai H., Igaki K. et al.; One year follow-up of biodegradable self-expanding stent implantation in humans; Journal of the American College of Cardiology, 37 (2001), 47A). It will be noted that the mechanical properties of stents of polymers, in particular based on biodegradable polymers, are markedly worse than metallic stents. The high level of flexural stiffness, the better recoil characteristic, better elongation at fracture and greater ease of processing are at the present time factors in favor of metallic stents with a polymeric coating instead of an implant of solid plastic. If the material poly-L-lactide is used as a volume material for the production of stents the known processing procedures involved (co-extrusion, injection molding etc.) result in very specific changes in the material properties, for example an increase in density and stiffness and a reduction in porosity. If in contrast the polymer is applied in the form of a coating material, not only are other material properties desired, but they already result from the greatly different manufacturing procedure (for example spraying or dipping process). Therefore the use of a polymer as a volume material does not make it possible to draw any conclusions about the properties of the same material as a coating.

For use on human beings, it is essential for the stent to be sterilized. Accordingly, to produce an implantable stent, a sterilization operation must always follow the polymeric coating operation. Current sterilization processes for polylactides, in particular the admitted processes which are known from the state of the art and consisting of steam sterilization, plasma sterilization with hydrogen peroxide and ethylene oxide sterilization result in a reduction in the molecular mass of the polymer and an in part considerable impairment in the stability in respect of shape of the coating. It is thought that a reason for this lies in the steps which are respectively involved for soaking the sterilization material as polylactides degrade under the action of water or hydrogen peroxide as a consequence of hydrolytic processes. Long exposure times in water-free processes such as gamma ray sterilization result in structural changes in the polymer due to radical formation. If drug-loaded coatings are sterilized then the above-mentioned processes also reduce the biological effectiveness of the active substances contained therein.

The object of the present invention is to provide an implantable stent having a polymeric coating. The coating material should admittedly bond to known materials, but by virtue of its properties it should enjoy improved compatibility and thus reduce inflammatory and proliferative processes which can result in restenosis. The invention further seeks to provide a process for the production of stents coated in that way, which satisfies the particular demands on the coating material.

### SUMMARY OF THE INVENTION

That object is attained by a stent having the features recited in the appended claims and an associated production process, also set forth in the claims. The fact that the polymeric coating in the implantable state after production and sterilization contains poly-L-lactide of a mean molecular weight of more than 200 kDa, in particular more than 350 kDa, makes it possible to evidently effectively suppress the restenosis-triggering factors. Surprisingly it was found that neointimal proliferation can be markedly reduced with such high-polymeric coatings. Evidently the use of the high-molecular polymer, in comparison with shorter-chain polymers, results in a marked reduction in inflammatory and proliferative processes.

The information relating to molecular weight, used in the sense according to the invention, relates to values which are determined in accordance with the Mark-Houwink (MH) formula. For the poly-L-lactide L214 used by way of example, from Boehringer Ingelheim, the molecular weight prior to sterilization is 691 kDa, according to the manufacturer's specification. After electron beam sterilization which is effected in the production process according to the invention, inter alia molecular weights of between 220 kDa and 245 kDa were determined, using the same process.

The high-molecular poly-L-lactide is suitable in particular as a drug carrier for pharmacologically active drugs. If therefore pharmacological therapy is additionally to be implemented at a local level, then one or more active substances can be embedded in per se known manner—at least being involved with the application of the polymeric coating.

Both in the case of the additional function as a drug carrier and also in sole use, a layer thickness of the polymeric coating is preferably between 3 and 30 μm, in particular between 8 and 15 μm. The selected ranges make it possible to ensure a sufficiently high degree of wetting of the surface of the stent. However, such thin coatings do not yet have a tendency to cracking and accordingly resist flaking detachment when the stent is subjected to a mechanical loading. Overall preferably between 0.3 and 2 mg, in particular between 0.5 and 1 mg, of coating material is applied per stent. In order to suppress inflammatory reactions the implant should be covered with the polymeric coating over as large a surface area as possible.

It is further advantageous if a base body of the implant is formed from at least one metal or at least one metal alloy. It is further advantageous if the metal or the metal alloy is at least partially biodegradable. The biodegradable metal alloy can be in particular a magnesium alloy. The stent, in the biodegradable variant, is completely broken down with time and this means that possible triggers for an inflammatory and proliferative reaction of the surrounding tissue also disappear.

In the case of active substance-loaded polymeric coatings, a stent design should preferably be so adapted that there is contact with the vessel wall over the largest possible surface area. That promotes uniform elution of the active substance which is substantially diffusion-controlled according to investigations. Regions of high mechanical deformability are preferably to be cut out in the coating as it is here that the risk of flaking detachment of the coating is increased. Alternatively or supplemental thereto the stent design can be so predetermined that, in the event of a mechanical loading, that is to say generally upon dilation of the stent, the forces occurring are distributed as uniformly as possible over the entire surface of the stent. It is possible in that way to avoid local overloading of the coating and thus crack formation or indeed flaking detachment of the coating.

The polymeric coating has a very high level of adhesion capability if the implant has a passive coating of amorphous silicon carbide. The polymeric coating can be applied directly to the passive coating. Alternatively it is possible to provide spacers or bonding layers which are bonded to the passive coating for further enhancing the adhesion capability of the polymeric coating.

In accordance with the process of the invention for the production of an implantable stent it is provided that the stent

(a) is wetted at least portion-wise with a fine mist of a solution of poly-Llactide of a mean molecular weight of more than 650 kDa,

(b) the solution applied to the stent is dried by blowing it away, and

(c) the stent is then sterilized by means of electron beam sterilization.

The operation of applying the polymeric coating is preferably effected by means of rotational atomizers which produce a finely distributed mist of very small suspended particles. For that purpose a solution of the high-molecular polymer, optionally mixed with one or more active substances, is withdrawn from a supply container. The fine spray mist causes surface wetting of very small structures of the implant and is then dried by being blown away. That operation can be repeated as desired until the desired thickness of the polymeric coating is achieved. Electron beam sterilization is then effected.

The sterilization process has proven to be particularly suitable for polylactides over admitted processes. Electron beam sterilization has no or only a slight influence on the stability in respect of shape of the polymeric coating and the biological effectiveness of a possibly embedded active substance. The exposure times in electron beam sterilization, which are only a few seconds long, prevent unwanted structural changes in the polymer due to radical formation. Admittedly, a marked reduction in molecular weight has to be tolerated, due to the sterilization procedure, but the operation can be controlled by presetting suitable parameters. Irradiation with a dosage in the range of between 15 and 35 kGy, in particular in the range of between 22 and 28 kGy, has proven to be particularly practicable, in a practical context. It is further preferable if the kinetic energy of the electrons is in the range of between 4 and 5 MeV. The reduction in molecular weight as a consequence of sterilization can be reduced with a falling dosage and/or falling kinetic energy of the electrons. Operating parameters for the sterilization procedure, which result in the setting of a specifically desired molecular weight, are to be ascertained in apparatus-specific fashion. In addition the operating parameters are also to be specified for the respective substrate, for variations in the properties of the polymeric coating such as for example the layer thickness and specific density thereof, which occur due to manufacture, also have an influence on the extent of the crack process. In general the reduction in molecular weight is decreased with increasing layer thickness and specific density of the coating.

Further preferred configurations of the invention will be apparent from the other features which are set forth in the appended claims.

### BRIEF DESCRIPTION OF THE DRAWINGS

The invention will be described in greater detail hereinafter by means of an embodiment and with reference to the drawings in which:

FIG. 1 shows a diagrammatic plan view of a portion of an endovascular implant in the form of a stent,

FIG. 2 is a view in section through a structural element of the stent with a polymeric coating, and

FIG. 3 shows a stent design which an alternative to FIG. 1.

### DETAILED DESCRIPTION OF A PREFERRED EMBODIMENT

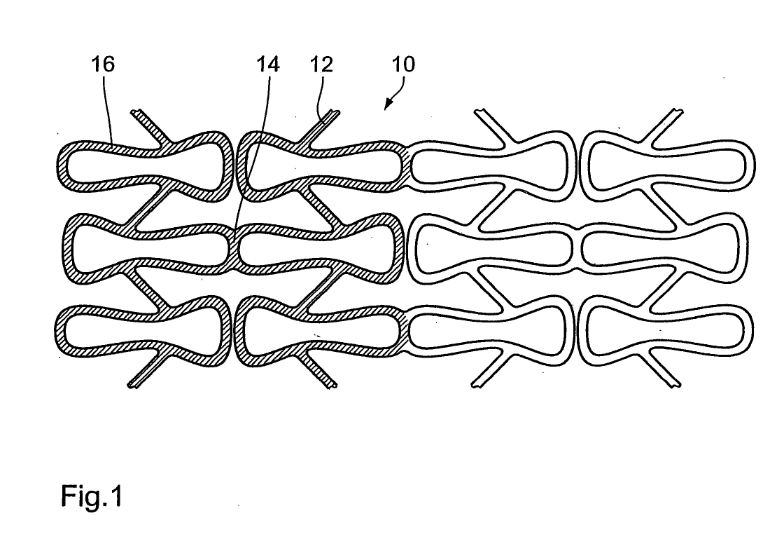

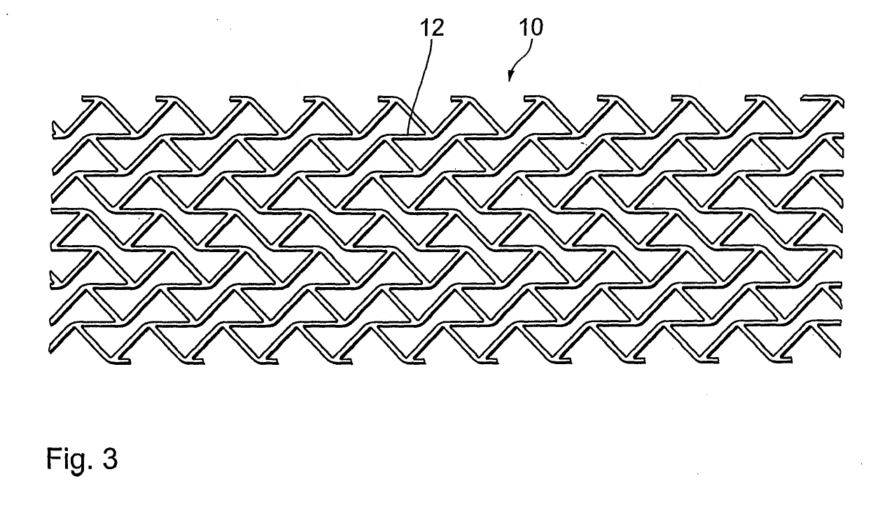

FIG. 1 is a diagrammatic view of a portion of an endovascular implant, here in the form of a stent 10. The stent 10 comprises a plurality of structural elements 12 which—as illustrated in this specific example—form a lattice-like pattern about the longitudinal axis of the stent 10. Stents of this kind have long been known in medical technology and, as regards their structural configuration, can vary to a high degree. What is of significance in regard to the present invention is that the stent 10 has an outwardly facing surface 14, that is to say a surface which is directed towards the vessel wall after implantation. In the expanded condition of the stent 10 that outward surface 14 should involve an area coverage which is as large as possible in order to permit uniform active substance delivery. In regard to the mechanical basic structure, distinctions are to be drawn in terms of the configuration involved: concentration of the deformation to a few regions or uniform deformation over the entire basic structure. In the former case, the structures are such that, upon mechanical expansion of the stent, there are only deformations concentrated in the region of flow hinges (thus for example in the stent 10 shown in FIG. 1). The second variant in which dilation results in deformation of virtually all structural elements 12 is shown by way of example in FIG. 3. It will be appreciated that the invention is not limited to the stent patterns illustrated. Modifications in the stent design which increase the contact surface area are generally preferred as, in the case of active substance-laden coatings, that permits more uniform elution into the vessel wall. In addition, regions involving a high level of mechanical loading, such as for example the flow hinges in FIG. 1 are either not to be coated or a stent design is predetermined (for example that shown in FIG. 3), which distributes the forces occurring upon dilation to all structures of the stent more uniformly. That is intended to avoid crack formation or flaking detachment of the coating as a consequence of the mechanical loading.

The surface 14 of the structural elements 12 is covered with a polymeric coating 16, indicated here by a surface with dark hatching. The polymeric coating 16 extends either over the entire surface 14 or—as shown here only over a portion of the surface 14. The polymeric coating 16 comprises poly-Llactide of a mean molecular weight of >200 kDa and involves layer thicknesses in the range of between 3 and 30 μm. The polymer is biocompatible and biodegradable. Degradation behavior on the part of the polymer can be influenced by a variation in the molecular weight, in which respect generally degradation time increases with increasing molecular weight of the polymer.

The polymeric coating 16 can also serve as a carrier for one or more pharmacologically active substances which are intended to be delivered to the surrounding tissue by way of the surface 14 of the structural elements 12. Active substances that are to be considered are in particular pharmaceuticals from the group of anti-coagulants, fibrinolytics, lipid reducers, antianginositics, antibiotics, immunosuppressives, cytostatics, PPAR-agonists, RXR-agonists or a combination thereof. Thus the polymeric coating 16 may contain in particular as the active substance a fibrate or a fibrate combination from the group of clofibrate, etofibrate, etofyllinclofibrate, bezafibrate, fenofibrate and gemfibrozil. Glitazones such as ciglitazone, pioglitazone, rosiglitazone and troglitazone as well as the RXR-agonists bexarotene and phytic acid are particularly suitable by virtue of their pharmacological action. The polymeric coating 16 permits controlled liberation of the active substances by diffusion or gradual degradation.

As the polymer is biodegradable the elution characteristic of the active substance can be influenced by varying the degree of polymerization. With a rising molecular weight for the polymer, the period of time in which the active substance is liberated also generally increases in length. The elution characteristic of a polymeric coating of that kind is preferably so adjusted that between 10 and 30% and in particular between 15 and 25% of the active substance is liberated within the first two days. The balance of the remaining active substance is to be successively delivered within the first months, also controlled by way of diffusion and degradation procedures.

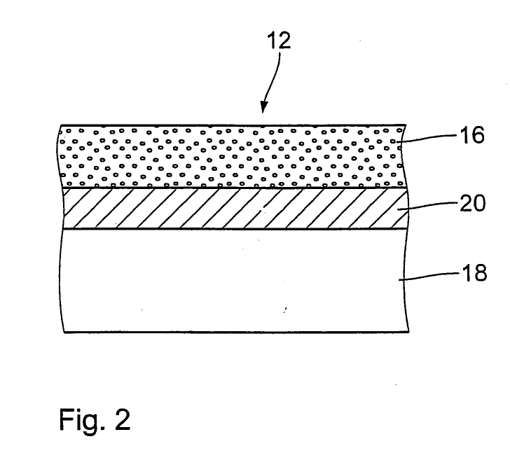

A particularly high degree of adhesion to the surface of the structural elements 12 can be achieved if the stent 10 at its surface 14 additionally has a passive coating 20 of amorphous silicon carbide (see FIG. 2). The production of structures of that kind is known from the state of the art, in particular from patent DE 44 29380 C1 to the present applicants, to the disclosure of which attention is directed in respect of the full extent thereof, and it is therefore not to be described in greater detail at this point. It merely remains to be emphasized that the adhesion capability of the polymeric coating material to the stent surface 14 can be improved with such a passive coating 20. In addition the passive coating 20 already reduces on its own neointimal proliferation.

A further improvement in the adhesion capability can be achieved if bonding of the polymer is effected covalently by means of suitable spacers or by applying a bonding layer. The essential traits of activation of the silicon carbide surface are to be found in DE 195 33682 A1 to the present applicants, to the disclosure of which attention is hereby directed in respect of the full extent thereof. The spacers used can be photoreactive substances such as benzophenone derivatives which, after reductive coupling to the substrate surface and possibly protection removal, provide functional binding sites for the polymer. A bonding layer which is a few nanometers thick can be achieved for example by silanization with epoxyalkylalkoxysilanes or epoxyalkylhalogen silanes and derivatives thereof. The poly-L-lactide is then bound to the bonding layer by physisorption or chemisorption.

FIG. 2 is a view in section through a structural element 12 of the stent 10 in any region thereof. The polymeric coating 16 is applied to a base body 18 with the above-mentioned passive coating 20 of amorphous silicon carbide. The base body 18 can be formed from metal or a metal alloy. If the entire stent 10 is to be biodegradable the base body 18 can be produced in particular on the basis of a biodegradable metal or a biodegradable metal alloy. A biodegradable magnesium alloy is particularly suitable. Materials of that kind are also already adequately described in the state of the art so that they will not be especially set forth here. In this connection attention is directed in particular to the disclosure in DE 198 56983 A1 to the present applicants.

Production of the polymeric coating 16 is implemented by means of a rotational atomizer which produces a mist of micro-fine particles. Alternatively it is also possible to use ultrasonic atomizers. The coating operation is effected stepwise in numerous cycles which comprise a step of wetting the stent in the spray mist produced and a subsequent step of drying the deposit on the stent by blowing it away. The multi-stage production process makes it possible to produce any layer thicknesses and—if desired—concentration gradients of the active substance or substances in individual layers of the polymeric coating 16. Sterilization of the stent is effected by electron bombardment, in which case partial cracking of the polymer chains by virtue of the high molecular weight of the polymer can be tolerated. The kinetic energy of the electrons is approximately in the range of between 4 and 5 MeV as, at those values, adequate sterilization with an only slight degree of depth of penetration into the base body 18 of the stent 10 is still ensured. The dosage ranges between 15 and 35 kGy per stent. Investigations showed that no or only a minimal reduction in the biological activity of embedded active substances occurred due to the sterilization process.

The layer thicknesses produced for the polymeric coating 16 are generally in the range of between 3 and 30 μm. Layer thicknesses in the range of between 8 and 15 μm are particularly desirable as that already ensures very substantial coverage of the surface 14 of the stent 10 and it is not yet necessary to reckon on the occurrence of structural problems such as crack formation and the like. Between about 0.3 and 2 mg, in particular between 0.5 and 1 mg, of coating material is applied per stent 10.

Embodiment:

A commercially available stent which can be obtained under the trade name LEKTON from BIOTRONIK is coated hereinafter with the polymer.

The stent was clamped in a rotational atomizer. A solution of poly-L-lactide of a mean molecular weight of 691 kDa in chloroform was prepared in a supply container (concentration: 7.5 g/l). The polymer can be obtained in the form of a granulate under the trade name RESOMER L214 from Boehringer Ingelheim. Clofibrate was used as the active substance.

The stent was wetted on both sides with a finely distributed mist produced by the rotational atomizer in 80 cycles each of a duration of about 10 s. The respective wetting operation was followed by a drying step by blowing-off of a duration of about 12 seconds. After the end of a total of 160 coating cycles the stent was removed. The layer thickness of the polymeric coating is about 10 μm and the mass of the polymeric coating is about 0.7 mg per stent.

After application of the coating electron beam sterilization of the stent is effected with 4.5 MeV-electrons at a dosage of 25 kGy. The sterilization operation reduced the mean molecular weight to about 230 kDa (determined using the Mark-Houwink method).

The implantable stent was tested in animal experiments on the cardiovascular system of a pig. For that purpose the stent was alternately implanted in the Ramus interventricularis anterior (RIVA), Ramus circumflexus (RCX) and the right coronary artery (RCA) of the heart of 7 pigs. For comparative purposes at the same time a blind test was started with stents without a coating. After 4 weeks the restenosis rates of the stents with and without polymeric coating were determined by measuring off the level of neointimal proliferation by means of quantitative coronary angiography and compared. There was a significant reduction in neointimal proliferation when using a stent with a polymeric coating.

What is claimed is:

1. A stent comprising:
an at least portion-wise polymeric coating, as measured in the implantable state after production and sterilization, of poly-L-lactide of a mean molecular weight of more than 200 kDa.

2. The stent of claim 1, wherein:
the mean molecular weight of the poly-L-lactide is more than 350 kDa.

3. The stent of claim 2, wherein:
a layer thickness of the polymeric coating is between 3 and 30 μm.

4. The stent of claim 3, wherein:
the layer thickness of the polymeric coating is between 8 and 15 μm.

5. The stent of claim 4, wherein:
the polymeric coating is on a base body of the stent that comprises at least one metal.

6. The stent of claim 4, wherein:
the polymeric coating is on a base body of the stent that comprises at least one metal alloy.

7. The stent of claim 6, wherein:
the metal alloy is at least partially biodegradable.

8. The stent of claim 7, wherein:
the biodegradable metal alloy is a magnesium alloy.

9. The stent of claim 5, wherein:
a passive coating containing amorphous silicon carbide is provided between the polymeric coating and the base body.

10. The stent of claim 9, wherein:
a spacer binds the polymeric coating to the passive coating.

11. The stent of claim 9, wherein:
a bonding layer binds the polymeric coating to the passive coating.

12. The stent of claim 11, wherein:
at least one pharmacologically active substance is contained in the polymeric coating.

13. The stent of claim 12, wherein:
the stent is adapted to maximize a contact surface with a vessel wall in which the stent would be placed.

14. The stent of claim 13, wherein:
the stent is adapted so that mechanical loading on the stent uniformly distributes the applied forces over all structural elements of the stent.

15. A process for producing an implantable stent with a polymeric coating of high-molecular poly-L-lactide, comprising the steps of:
(a) wetting the stent at least portion-wise with a fine mist of a solution of poly-L-lactide of a mean molecular weight of more than 650 kDa;
(b) drying the solution applied to the stent by blowing; and
(c) sterilizing the stent with electron beam sterilization.

16. The process of claim 15, wherein:
the process steps of wetting and drying are repeated until the polymeric coating is of a layer thickness of between 3 and 30 μm.

17. The process of claim 16, wherein:
the electron beam sterilization is implemented with a dosage in the range of between 15 and 35 kGy.

18. The process of claim 17, wherein:
the dosage is in the range of between 22 and 28 kGy.

19. The process of claim 16, wherein: the electron beam sterilization is conducted with a predetermined electron kinetic energy in the range of between 4 and 5 MeV.

20. The stent of claim 1, wherein:
a layer thickness of the polymeric coating is between 3 and 30 μm.

21. The stent of claim 20, wherein:
the layer thickness of the polymeric coating is between 8 and 15 μm.

22. The stent of claim 1, wherein:
the polymeric coating is on a base body of the stent that comprises at least one metal.

23. The stent of claim 1, wherein:
the polymeric coating is on a base body of the stent that comprises at least one metal alloy.

24. The stent of claim 5, wherein:
the metal is at least partially biodegradable.

25. The stent of claim 5, wherein:
the metal alloy is at least partially biodegradable.

26. The stent of claim 5, wherein:
the metal is at least partially biodegradable.

27. The stent of claim 25, wherein:
the biodegradable metal alloy is a magnesium alloy.

28. The stent of claim 22, wherein:
a passive coating containing amorphous silicon carbide is provided between the polymeric coating and the base body.

29. The stent of claim 6, wherein:
a passive coating containing amorphous silicon carbide is provided between the polymeric coating and the base body.

30. The stent of claim 23, wherein:
a passive coating containing amorphous silicon carbide is provided between the polymeric coating and the base body.

31. The stent of claim 28, wherein:
a spacer binds the polymeric coating to the passive coating.

32. The stent of claim 29, wherein:
a spacer binds the polymeric coating to the passive coating.

33. The stent of claim 30, wherein:
a spacer binds the polymeric coating to the passive coating.

34. The stent of claim 28, wherein:
a bonding layer binds the polymeric coating to the passive coating.

35. The stent of claim 29, wherein:
a bonding layer binds the polymeric coating to the passive coating.

36. The stent of claim 30, wherein:
a bonding layer binds the polymeric coating to the passive coating.

37. The stent of claim 1, wherein:
at least one pharmacologically active substance is contained in the polymeric coating.

38. The stent of claim 1, wherein:
the stent is adapted to maximize a contact surface with a vessel wall in which the stent would be placed.

39. The stent of claim 1, wherein:
the stent is adapted so that a mechanical loading on the stent uniformly distributes the applied forces over all structural elements of the stent.

40. The process of claim 15, wherein:
the electron beam sterilization is implemented with a dosage in the range of between 15 and 35 kGy.

41. The process of claim 40, wherein:
the dosage is in the range of between 22 and 28 kGy.

42. The process of claim 15, wherein: the electron beam sterilization is conducted with a predetermined electron kinetic energy in the range of between 4 and 5 MeV.
